(19) United States
(12) Patent Application Publication
(10) Pub. No.: US 2009/0062232 A1
(43) Pub. Date: Mar. 5, 2009

(54) High-Purity Xylooligosaccharide Compositions

(75) Inventors: Shigeaki Fujikawa, Osaka (JP); Hiroaki Sasaki, Kyoto (JP); Tadayoshi Ishizuka, Hokkaido (JP)

Correspondence Address: DRINKER BIDDLE & REATH (DC), 1500 K STREET, N.W., SUITE 1100, WASHINGTON, DC, 20005-1209, US

(73) Assignees: SUNTORY LIMITED, Osaka-shi, Osaka (JP); HOKKAIDO SUGAR CO., LTD., Tokyo (JP)

(21) Appl. No.: 11/918,402

(22) PCT Filed: Apr. 14, 2006
(86) PCT No.: PCT/JP2006/307910

§ 371 Date: Oct. 12, 2007

(30) Foreign Application Priority Data
Apr. 15, 2005 (JP) 2005-119032

(51) Int. Cl. A61K 31/702; C12P 19/00; C07H 3/06; A23L 1/48; A23L 2/38

(52) U.S. Cl. 514/54; 435/72; 536/123.1; 426/634; 426/590

(57) ABSTRACT

To provide a high-purity xylooligosaccharide composition while preventing the formation of UV-absorbing substances and coloring matters.

A high-pure xylooligosaccharide composition containing reduced UV-absorbing substances and reduced coloring matters and a method of producing the same which comprises alkali-treating or pressure- and heat-treating a plant material such as wood, corn cob, cotton seed hull, bagasse or rice straw, further enzymatically treating the same, and purifying the crude saccharide solution containing the residues thus obtained, wherein the solution is concentrated and then optionally subjected to desalting and active carbon-treatment. According to the method of the invention, the saccharified solution is concentrated followed by the desalting and active-carbon treating so that a high-purity xylooligosaccharide composition can be produced while preventing the formation of UV-absorbing substances and coloring matters.

### FIELD OF INDUSTRIAL APPLICATION

This invention relates to methods of obtaining high-purity xylooligosaccharides containing reduced UV-absorbing substances and reduced coloring substances which comprise, in the process comprising pretreating a plant material selected from the group consisting of wood, corn cob, cotton seed hull, bagasse and rice straw and saccharifying to give a xylooligosaccharide solution, efficiently separating a crude saccharide solution obtained by the saccharification into solid and liquid and decoloring.

### PRIOR ART

### Use of Xylooligosaccharides

Oligosaccharides, which are characterized by having a bifidus activity (an effect of improving the bacterial flora in the intestine) in addition to favorable properties of, for example, having a low sweetness, a low caloric value and a reduced caries-producing effect, have been employed in a large number of marketed products including foods for specified health uses declaring the effect of regulating intestinal functions. Among these oligosaccharides, xylooligosaccharides have the following characteristics. That is, since a xylooligosaccharide is hardly decomposed by acids or digestive enzymes such as amylase, it gets to the large intestine without decomposed and without absorbed when taken by humans. In the large intestine, the xylooligosaccharide is selectively utilized by bifidobacteria inhabiting there. Thus, the xylooligosaccharide contributes to the growth of the bifidobacteria even in a small amount and, as a result, improves bowel movement and promotes Ca absorption. Therefore, xylooligosaccharides are widely applicable to foods and so on.

### Fundamental Method of Producing Xylooligosaccharides

With the advances and development of the enzymatic chemistry, a large number of microbial-origin hydrolases and transferases have been found out. As the results of further intensive studies, moreover, various oligosaccharides have been economically produced on a mass scale. Owing to the development of highly active xylanase, in particular, it becomes possible to produce xylooligosaccharides, which are excellent in physical properties and functions among oligosaccharides, from xylan which is a hemicellulose contained in a large amount in plant materials having been scarcely utilized hitherto such as wood, corn cob, cotton seed hull, bagasse and rice straw.

Examples of the existing techniques for producing xylooligosaccharides from plant materials are as follows.

(1) A method of directly producing a xylooligosaccharide solution by a saccharification treatment such as pressure- and heat-treating, blasting or alkali-treating.

(2) A method of producing a xylooligosaccharide, which comprises xylotetraose as the main component and has a high average degree of polymerization of 5.4, by acid-treating lignocellulose obtained from chemical pulp.

(3) A method of producing a xylooligosaccharide solution starting with xylan, which is obtained by pressure- and heat-treating, alkali-treating under heating, extracting or purifying, and saccharifying it by treating with an enzyme.

(4) A method of producing a xylooligosaccharide solution by cutting a plant material into small pieces, alkali-treating under heating, saccharifying by directly treating with an enzyme and then separating into solid and liquid.

For example, Kusakabe et al. (J. Japan Soc. of Agrochem., Vol. 50, No. 5, p. 209-215, 1976) produced a xylooligosaccharide by pretreating corn cob employed as the starting material with an alkali, removing the alkali by washing with water until the pH value attained neutrality and then hydrolyzing with the use of a bacillus-origin enzyme.

### Need for High-Purity Xylooligosaccharides

In the case of using in a processed food or drink, it is desirable that a xylooligosaccharide is colorless to elevate the degree of freedom in processing the processed food or drink. In producing a processed food or drink, moreover, it has been a common practice to conduct a sterilization treatment at a high temperature for killing microorganisms. It is known that saccharides are colored upon heating and xylooligosaccharides show a particularly strong tendency toward the coloration. In xylooligosaccharides which is a general name indicating oligosaccharides having degree of polymerization of 2 or more, obvious coloration is observed in xylooligosaccharides having low degree of polymerization. That is to say, the tendency of xylooligosaccharides toward coloration is weakened with an increase in the degree of polymerization. In xylooligosaccharides having high degree of polymerization, however, metabolic properties by bifidobacteria and lactic acid bacteria inhabiting the large intestine are worsened (Okazaki et al., Bifidobacteria Microflora Vol. 9, p. 77 1990). Accordingly, there has been required a xylooligosaccharide comprising xylobiose having a degree of polymerization of 2 as the main component. Thus, there has been required a xylooligosaccharide which is not only colorless but also undergoes reduced coloration upon a heat treatment at a high temperature.

However, a crude saccharide solution obtained by saccharification by any one of the methods (1) to (4) as described above contains various impurities and residues. To remove these contaminants, it has been a practice to purify the crude saccharide solution by filtration or using an adsorbent such as an ion exchange resin, a synthetic adsorbent or active carbon. In particular, a saccharide solution having been hydrolyzed with, for example, an enzyme contains a considerably large amount of impurities extracted from the plant material such as lignin and these impurities can be hardly removed by the filtration procedure commonly employed. To solve this problem, there have been proposed various methods, for example, a method of removing coloring components with the use of active carbon or an ion exchange resin.

### Purification Method Using Active Carbon or Ion Exchange Resin

Japanese Patent No. 3229944 (Forestry Agency and TOWAKASEI Co., Ltd.) discloses a method comprising steaming cotton seed hull, enzymatically decomposing it, and deionizing the resulting crude saccharide solution containing xylooligosaccharide by treating with active carbon. However, it discloses no method of inhibiting the formation of UV-absorbing substances such as furfural produced thereby. This method is to be used in the case of using steamed cotton seed hull. Namely, no method of purifying a crude saccharide solution originating in a material containing much high-molecular weight coloring components is disclosed therein.

In JP-A-2001-226409 (OJI PAPER Co., Ltd.), enzymatically delignified pulp obtained from hardwood chips was treated with xylanase and then decomposed with sulfuric acid to give a crude xylooligosaccharide solution having high degree of polymerization. Then this solution was concentrated, treated with an ion exchange resin and then treated with active carbon to give a xylooligosaccharide with high degree of polymerization comprising xylotetraose (X4) and xylopentaose (X5) as the main components which showed no absorption at 280 nm and 250 nm and had reduced ash content. In this method, the saccharified solution containing lignin in an already lowered amount was further treated with an ion exchange resin and active carbon to give a solution showing no absorption at 280 nm and 250 nm. Moreover, the oligosaccharide thus formed was a xylooligosaccharide with high degree of polymerization comprising X4 and X5 as the main components. Namely, the content of xylose (monosaccharide) in the total saccharides was as low as 8.37% and thus furfural showing absorption at 280 nm was scarcely formed therein compared with a xylooligosaccharide with low degree of polymerization.

### Other Purification Methods

In producing a xylooligosaccharide by an enzymatic reaction with the use of a plant material such as corn cob, cotton seed hull, bagasse or rice straw, the starting material should be pretreated by, for example, alkali-treating or pressure- and heat-treating so as to efficiently form the xylooligosaccharide by the enzymatic reaction. In the case of using corn cob, however, a saccharified solution having been thus pretreated is contaminated with water-soluble high-molecular weight impurities remaining therein.

To remove these water-soluble high-molecular weight impurities, there have been proposed a method wherein an UF membrane is used (TOWAKASEI Co., Ltd.: JP-A-61-285999), a method wherein the impurities are converted into organic acids by oxidation with the use of ozone and then adsorbed by an ion exchange resin (TOWAKASEI Co., Ltd.: JP-A-62-281890) and so on. In the method of purifying with the use of an UF membrane, however, residues contained in the crude saccharide solution cause clogging in the UF membrane and thus filtration should be preliminarily conducted until the solution becomes clear. On the other hand, the method using the ozone treatment suffers from some troubles such that it is a time-consuming method and yet sufficient removal effect can be hardly achieved thereby. Under these circumstances, SUNTORY, Ltd. et al. (JP-A-5-253000) proposed a method comprising subjecting small pieces of a plant material such as corn cob successively to an alkali-treatment, washing with water and enzymatic saccharification reaction, and then adding lime and carbon dioxide gas to the reaction mixture without separating the residues to form insoluble lime carbonate salt followed by filtration. Thus, they found out that the filtration could be favorably conducted, the yield could be elevated with the use of a lessened amount of washing water and the impurities such as water-soluble polymers could be sufficiently removed, thereby elevating the purity of the saccharide. According to this document, the obtained saccharide solution could be purified by decoloring with active carbon or an ion exchange resin, desalting with an ion exchange resin followed by, if necessary, passing through a sterilization filter and concentrating to obtain a commercialized product.

### Need for High-Purity Xylooligosaccharides

On the other hand, it is preferred that the purity of a xylooligosaccharide solution is as high as possible in order to prevent the solution from the proliferation of microorganisms therein during storage, avoid damages in the inherent composition of a food to which the xylooligosaccharide solution is to be added, and reduce the transportation cost. In the case of producing a powdered xylooligosaccharide, it is desirable to use a concentrated solution for spray drying.

However, a xylooligosaccharide solution obtained by enzymatic decomposition or steam decomposition has a low saccharide concentration. In the method of JP-A-5-253000 as described above, for example, the cleaned saccharide solution, from which impurities such as water-soluble polymers had been sufficiently removed, had a low Brix of 2.61. Moreover, this xylooligosaccharide solution had a high salt concentration and low-molecular weight coloring components still remained therein. Therefore, such a solution should be further decolored, purified and concentrated before putting on the market.

Thus, the cleaned saccharide solution should be concentrated. In the course of concentrating the solution after the decoloration with active carbon or an ion exchange resin or desalting with an ion exchange resin, however, xylose and the like contained in the xylooligosaccharides are liable to undergo coloration, compared with other hexoses such as glucose and sucrose, and UV-absorbing impurities such as furfural are formed thereby. Thus, the solution is further colored and impurities are increased. Concentration in the acidic region at a high temperature would frequently cause the formation of UV-absorbing impurities such as furfural and coloration, while concentration in the alkaline region at a high temperature would frequently cause the decomposition of the xylooligosaccharides, the formation of coloring matters and lowering in the concentration efficiency due to a serious lowering in the heat transfer efficiency caused by the deposition of Ca salt tank stones on the concentration tank. To avoid these troubles by maintaining the pH value at neutrality, it is required to use a large amount of an alkali or an acid. After the completion of the concentration, desalting should be conducted by using a large amount of an ion exchange resin to remove such an alkali or acid. As a result, not only the cost is elevated but also the xylooligosaccharide yield is lowered in the desalting step.

In producing oligosaccharide for edible uses, it is desirable that plant hemicellulose, which has been eaten for a long time, is used as the starting material and the starting material can be easily obtained. From these viewpoints, development of xylooligosaccharides starting from corn cob is preferred to cotton seed chaff or wood chips. However, no method has been known hitherto for producing a xylooligosaccharide, which comprises xylobiose as the main component, has low degree of polymerization and contains reduced UV-absorbing substances or reduced coloring matters, starting with a saccharified solution containing high-molecular weight coloring matters and UV-absorbing substances such as a corn cob saccharified solution.
[Patent Document i] Japanese Patent No. 3229944
[Patent Document 2] JP-A-2001-2264090
[Patent Document 3] JP-A-61-285999
[Patent Document 4] JP-A-62-281890
[Patent Document 5] JP-A-5-253000
[Non-patent Document 1] J. Japan Soc. of Agrochem., Vol. 50, No. 5, p. 209-215, 1976
[Non-patent Document 2] Bifidobacteria Microflora, Okazaki et al., Vol. 9, p. 77

### DISCLOSURE OF THE INVENTION

### Problems that the Invention is to Solve

An object of the invention is to provide a method of producing a high-purity xylooligosaccharide starting with a plant material such as corn cob, cotton seed hull, bagasse or rice straw, which comprises pretreating the material by an alkali-treatment or pressure- and heat-treating, and further efficiently removing high-molecular weight water-soluble impurities remaining in the saccharified crude saccharide solution to thereby give a high-purity xylooligosaccharide contaminated with reduced UV-absorbing substances or reduced coloring matters.

Another object of the invention is to provide a method of producing a high-purity xylooligosaccharide, which is contaminated with reduced UV-absorbing substances or reduced coloring matters and contains xylooligosaccharides having degree of polymerization of from 2 to 3 in a large amount, by the above-described method of producing a xylooligosaccharide.

Another object of the invention is to provide a method of producing a high-purity xylooligosaccharide, which is contaminated with reduced UV-absorbing substances or reduced coloring matters and suffers from the formation of reduced UV-absorbing substances or reduced coloring matters even in the case of being steam-concentrated to give a solid saccharide content of from 30% to 75%, by the above-described method of producing a xylooligosaccharide.

Another object of the invention is to provide a method of producing a high-purity xylooligosaccharide, which is contaminated with reduced UV-absorbing substances or reduced coloring matters, contains xylooligosaccharides having degree of polymerization of from 2 to 3 in a large amount and suffers from the formation of reduced UV-absorbing substances or reduced coloring matters even in the case of being steam-concentrated to give a solid saccharide content of from 30% to 75%, by the above-described method of producing a xylooligosaccharide.

Moreover, an object of the invention is to provide a high-purity xylooligosaccharide contaminated with reduced UV-absorbing substances or reduced coloring matters which is produced by the method according to the invention.

### Means for Solving the Problems

To overcome the problems as discussed above, the inventors conducted intensive studies. As a result, they found out that a stronger coloration arose in heating a xylooligosaccharide at a high temperature at the larger content of substances absorbing UV light (UV-absorbing substances) contaminating the xylooligosaccharide. Under these circumstances, the inventors studied on a method of elevating the efficiency of removing these UV-absorbing substances. Consequently, they have found out that a xylooligosaccharide less contaminated with UV-absorbing substances and coloring matters can be obtained by alkali-treating or pressure-treating a plant material selected from the group consisting of wood, corn cob, cotton seed hull, bagasse and rice straw (preferably in the form of small pieces), further treating it with an enzyme, filtering the crude saccharide solution thus obtained to remove solid matters and concentrating followed by desalting and/or active carbon-treating, compared with the case where the same procedures are conducted without concentrating, thus completing the present invention.

### Preparation of Crude Saccharide Solution

As the plant material to be used in the method of the invention, one or more materials selected from among wood, corn cob, cotton seed hull, bagasse, rice straw and so on may be cited. The effect of the method of the present invention is particularly obvious in the case of producing a xylooligosaccharide starting with corn cub the crude saccharide solution of which can be hardly decolored.

The starting material can be pretreated by dipping in an alkali solution and subjecting to a high-temperature treatment, a high-temperature high-pressure treatment or a lignin-decomposing enzyme treatment. The alkali-treatment can be conducted by using, for example, caustic soda, ammonia, etc. In the case of carrying out the pretreatment with the use of a lignin-decomposing enzyme, the treatment can be carried out under the optimum conditions for the enzyme.

As the enzyme for enzymatically treating the pretreated material, use may be made of those capable of forming xylooligosaccharides of low degree of polymerization, which are exemplified by xylobiose and xylotriose, as the main components. Xylanase is a typical example of such enzymes. Known examples of the xylanase include those produced by a bacterium Bacillus subtilis, a ray fungus Streptomyces sp. and fungi belonging to the genera Aspergillus, Trichoderma, Penicillium Claudosporium and so on. From these enzymes, an appropriate one is selected and employed depending on the purpose. The enzyme treatment is conducted under such conditions as enabling the production of the desired xylooligosaccharide comprising xylobiose as the main component (i.e., the content amounting to 20% by weight or more based on the total saccharides). By employing these conditions, a crude saccharide solution comprising xylobiose as the main component and/or containing 30% by weight or less or 5% by weight or less of monosaccharides, based on the total saccharides, can be obtained. A person skilled in the art can easily adjust and optimize these conditions.

It is favorable, though not essentially required, to filter off the solid matters contained in the crude saccharide solution after treating with the enzyme. For the filtration, use can be made of diatomaceous earth filtration. It is particularly preferred to conduct the filtration as follows. Namely, lime is added to the crude saccharide solution containing the residues obtained after the enzyme treatment and further carbon dioxide gas is added thereto to form an insoluble lime salt followed by the filtration. As a substitute for the carbon dioxide gas, use may be made of an arbitrary acid capable of reacting with lime to form an insoluble lime salt, for example, oxalic aid or phosphoric acid. By using the formation of the lime salt, the filtration can be efficiently carried out while preventing the filter membrane from clogging.

Concentration of Crude Saccharide Solution and Purification after Concentration

An important characteristic of the method according to the invention resides in purifying the crude saccharide solution, from which the solid matters have been removed by the filtration, by the optimized combination of (1) desalting, (2) concentration and (3) active carbon-treatment to thereby give a high-purity xylooligosaccharide composition.

In the case where the salt precipitation should be prevented during the concentration, the crude saccharide solution is first desalted to thereby lower the salt concentration and then the pH value is adjusted to around neutrality before the concentration. The desalting may be conducted by a commonly employed method with the use of a cation exchange resin and/or an anion exchange resin.

By preliminarily concentrating the crude saccharide solution to a certain extent or concentrating to the final concentration and then conducting the desalting and/or active-carbon treatment, an enhancement in the coloration and an increase in the UV-absorbing substances accompanying the heat concentration can be suppressed. At the same time, the active-carbon treatment of the saccharide solution at a high saccharide concentration contributes to the decoloration and improvement in the efficiency of removing the UV-absorbing substances. In the method of the invention, namely, a purified saccharide solution is not concentrated to the desired saccharide concentration but a crude saccharide solution is concentrated and then purified. Thus, the formation of the UV-absorbing substances can be inhibited during the concentration (in particular, steam concentration) and, furthermore, the removal of the UV-absorbing substances and decoloration can be conducted at higher efficiencies in the concentrated crude saccharide solution. These facts will be indicated in practice in EXAMPLE 3.

Although it is preferred to conduct the concentration so that the saccharide concentration (solid concentration) comes to the marketed product level as close as possible, the viscosity is elevated at an excessively high concentration and the handling properties in the subsequent active-carbon treatment are worsened. In the case where the concentration is insufficient, on the other hand, the active-carbon treatment efficiency and the ion exchange resin-treatment efficiency are lowered and enhanced coloration and an increase in the UV-absorbing substances are induced by the subsequent heat concentration. Before conducting the decoloration with the use of active carbon or an ion exchange resin, therefore, the crude saccharide solution is concentrated to give a solid concentration of from 40 to 75%, preferably from 45 to 65%. Thus, lignin components can be removed by the subsequent purifying procedure and a high-purity xylooligosaccharide containing reduced UV-absorbing substances formed in the concentration step and scarcely suffering from coloration can be obtained. The solid concentration can be easily determined by evaporating the moisture. Alternatively, it is convenient to measure it with a Brix meter.

The crude saccharide solution can be concentrated by a method common employed for concentrating saccharide solutions. For example, it can be concentrated by steaming at a temperature around the boiling point under atmospheric or reduced pressure. As a concentration apparatus, use can be made of, for example, a multi-purpose tank. It is more preferred to conduct the concentration under reduced pressure.

For purifying the concentrated crude saccharide solution, the active carbon-treatment, the cation exchange resin-treatment and an anion exchange resin-treatment may be carried out at an arbitrary order. As the active carbon, any active carbon usable in purifying foods can be employed. The term “active carbon” as used herein has the same meaning as “absorbent”. Therefore, use may be made of an adsorbent, for example, a synthetic adsorbent such as graphite carbon or styrene divinylbenzene polymer as a substitute for the active carbon. As the ion exchange resin to be employed, it is possible to use a strongly acidic cation exchange resin, a weakly alkaline anion exchange resin or a mixed bed type exchange resin comprising a cation exchange resin with an anion exchange resin.

By purifying the concentrated crude saccharide solution, the UV-absorbing substances and the coloring matters can be efficiently removed. The removal of these substances can evaluated by measuring the absorbances at 280 nm and 230 nm assignable to the UV-absorbing substances and at 420 nm assignable to the coloring matters and confirming lowering in these absorbances compared with the values before the purification.

### ADVANTAGES OF THE INVENTION

According to the invention, the formation of UV-absorbing impurities and coloring matters can be inhibited by desalting and concentrating a crude saccharide solution to a certain concentration before decoloring with the use of active carbon or an ion exchange resin. Thus, the crude saccharide solution of xylooligosaccharide, etc. can be efficiently cleaned and a purified xylooligosaccharide product having a high purity and containing reduced impurities can be obtained. Moreover, it is possible to prevent products with the use of the xylooligosaccharide from coloration.

Because of being reduced colored, the xylooligosaccharide produced by the method according to the invention can be added to processed foods, drinks, health foods, supplements, foods for specified health uses, cosmetics, pet foods and so on and thus products with high qualities can be produced.

Next, the present invention will be illustrated in greater detail by referring to the following EXAMPLES. However, it is to be understood that the invention is not restricted to these EXAMPLES but involves production methods with the use of the idea thereof in its scope.

### BEST MODE FOR CARRYING OUT THE INVENTION

Examples of the modes for embodying the present invention are as follows.

i) After the completion of the enzymatic saccharification, impurities such as colorants are removed from the saccharified solution by the carbonic acid-saturation treatment, membrane separation, ion exchange resin-treatment or active carbon-treatment. Then the salt concentration is lowered and the pH value is adjusted at around neutrality. After preliminarily concentrating the solution to a certain extent, it is further subjected to desalting and active carbon-treatment to thereby finally give a xylooligosaccharide syrup having a saccharide concentration of 75%. By diluting this syrup to give a saccharide concentration of 37.5%, a saccharide solution with reduced UV-absorbing contaminants, which shows an absorbance at 420 nm in a 5 cm cell of 0.2 (preferably 0.06); or absorbances at 280 nm and 230 nm in a 1 cm cell of 1.28 or less and 3.7 or less respectively, is obtained. (The concentration of 50% in EXAMPLE 3 with the addition of 1% of active carbon is converted into 37.5%).

ii) After the completion of the enzymatic saccharification, the salt concentration is lowered or impurities such as colorants in the saccharified solution are decreased to a certain degree by the carbonic acid-saturation treatment, membrane separation, active carbon-treatment or desalting. Then the pH value is adjusted at around neutrality. After preliminarily concentrating the solution to a certain extent, it is further subjected to desalting. Next, monosaccharides are removed by chromatography with the use of an ion exchange resin and the residue is treated with active carbon and spray-dried to finally give a xylooligosaccharide powder having a moisture content of 6% or less and a monosaccharide content of 5% or less. A solution prepared by dissolving this powder in water to give a concentration of 20% shows a color tone of an absorbance at 420 nm in a 5 cm cell of 0.1 (preferably 0.05); or absorbances at 280 nm and 230 nm in a 1 cm cell of 1 or less and 2 or less respectively. That is, a saccharide solution containing reduced UV-absorbing impurities can be obtained.

### EXAMPLE 1

### Production of High-Purity Xylooligosaccharide Solution

(1) Small corn cob pieces were dipped in hot water containing caustic soda dissolved therein and stirred while maintaining at 90° C. for 90 minutes. Then, the mixture was filtered and washed with hot water to thereby remove the alkali until the pH value attained 11 or less.

(2) To the pretreated solid matter, water was added and the pH value was adjusted to 5.6 with the use of sulfuric acid or sodium hydroxide. Next, an enzyme xylanase was added thereto and an enzyme reaction was performed at 46° C. for 12 hours.

(3) While maintaining the enzyme reaction mixture at a liquid temperature of about 46° C., 40% by weight or more, based on the starting corn cob material, of lime milk (CaO) was continuously added to the saccharification reaction mixture. Then, carbon dioxide gas was blown thereinto to control the pH value to about 8.5. After the completion, the mixture was immediately filtered to give a clear saccharide solution.

(4) This clear filtrate was successively passed through a cation exchange resin (MITSUBISHI DIAION PK-216) and an anion exchange resin (MITSUBISHI DIAION WA-30). The saccharide concentration of the desalted solution was Brix 2.2%. It is preferable that the desalted solution has a pH value of from 4 to 7. In the case where the solution became alkaline, the pH value was adjusted to 4 to 7 by adding sulfuric acid.

(5) The desalted liquor, the pH value of which had been thus adjusted to 4 to 7, was concentrated to give a saccharide concentration of about Brix 20% by using a multipurpose tank.

(6) The concentrated solution was further desalted by using a mixed bed type ion exchange resin (MITSUBISHI DIAIONS PK16, PA412).

(7) Then, the solution was concentrated until the saccharide concentration attained about Brix 50%. This solution showed a pH value of about 6.5.

(8) The concentrated solution was further passed through a mixed bed type ion exchange resin (MITSUBISHI DIAIONS PK16, PA412). Next, 2% by weight, based on the total solid saccharide content, of active carbon was added thereto and the solution was treated for 1 hour. Then, the active carbon was removed by adding diatomaceous earth and filtering.

(9) Subsequently, the filtrate was concentrated to Brix 74.5, thereby giving a high-purity xylooligosaccharide solution.

The saccharide composition of the xylooligosaccharide solution thus obtained was as follows: xylose 23.4%, glucose 4.5%, xylobiose 34.4%, cellobiose 3.0%, xylotriose 8.51%, xylotetraose or oligosaccharides having higher polymerization degrees 25.7%.

To evaluate the color tone, the saccharide solution was diluted to saccharide concentrations of 50% and 37.5% and the absorbances at 420 nm were measured in a 5 cm cell. As a result, the absorbances were respectively 0.07 and 0.06, i.e., being almost colorless. When absorbances of these solutions were measured with the use of a 1 cm cell, the absorbances at 280 nm were 1.1 and 0.85 while the absorbances at 230 nm were 3.2 and 2.5, indicating that the saccharide solution contained reduced UV-absorbing impurities. The content of furfural in the solution of the 50% saccharide concentration was 5 ppm.

TABLE 1

| | 420 nm | 280 nm | 230 nm |
|---|---|---|---|
| | (5 cm cell) | (1 cm cell) | (1 cm cell) |
| Absorbance of saccharide | 0.07 | 1.1 | 3.2 |
| solution with 50% saccharide | | | |
| concentration | | | |
| Absorbance of saccharide | 0.06 | 0.85 | 2.5 |
| solution with 37.5% | | | |
| saccharide concentration | | | |

### EXAMPLE 2

### Production of High-Purity Xylooligosaccharide Solution

The saccharide solution, which had been treated as in EXAMPLE 1 until the first treatment by using the mixed bed type ion exchange resin and the subsequent concentration to give a saccharide concentration of about Brix 50%, was further treated with the mixed bed type ion exchange resin and then monosaccharides such as xylose were removed therefrom by ion chromatography to thereby give a xylooligosaccharide solution with a monosaccharide content of 5% or less. This solution was subjected to the same active carbon-treatment as in EXAMPLE 1 and then the active carbon was removed by adding diatomaceous earth and filtering. By spray-drying this solution, a high-purity xylooligosaccharide powder having a moisture content of 6% or less could be produced.

The saccharide composition of this powder was as follows: xylose 0.67%, xylobiose 33.2%, xylotriose 13.78%, xylotetraose or oligosaccharides having higher polymerization degrees 46.29%, cellobiose 4.4%, monosaccharides such as glucose 1.59%.

This powder was dissolved in purified water to give a saccharide concentration of 20 g/100 ml and the color tone was evaluated. As a result, the absorbance at 420 nm measured in a 5 cm cell was 0.03, i.e., being almost colorless. When absorbances were measured with the use of a 1 cm cell, the absorbances at 280 nm and 230 nm were respectively 0.20 and 1.30, indicating that the saccharide solution contained reduced UV-absorbing impurities. The content of furfural in the solution of the 20% saccharide concentration was 3 ppm.

TABLE 2

| | 420 nm | 280 nm | 230 nm |
|---|---|---|---|
| | (5 cm cell) | (1 cm cell) | (1 cm cell) |
| Absorbance of saccharide | 0.03 | 0.2 | 1.3 |
| solution with 20% saccharide | | | |
| concentration | | | |

### EXAMPLE 3

### Effect of Active-Carbon Treatment on High-Concentration Saccharide Solution

The saccharide solution, which had been treated as in EXAMPLE 1 until the first desalting by using the mixed bed type ion exchange resin (MITSUBISHI DIAIONS PK216, PA412) and the subsequent concentration to give a solid concentration of 50%, was further diluted 10-fold by weight to give a solution containing 5% of solid matters. To the solution having a solid concentration of 50%, active carbon was added to give weight ratios to the solid matters of 2, 4 and 8% (weight ratios to the solution of 1, 2 and 4% respectively). To the solution having a solid concentration of 5%, active carbon was added to give weight ratios to the solid matters of 2, 4 and 8% (weight ratios to the solution of 0.1, 0.2 and 0.4% respectively). Then, these solutions were stirred at 50° C. for 60 minutes and filtered.

At the same addition level of active carbon, as a result, the removal ratios represented by the absorbances at 420 nm, 280 nm and 230 nm of the solution of a solid concentration of 50% were higher. That is to say, the efficiency of removing the UV-absorbing substances and the coloring matters can be elevated by conducting the active-carbon treatment at a higher concentration.

When the saccharide solution of a solid concentration of 5% having been treated with active carbon was heated at 100° C. for 30 minutes, the absorbances at 420 nm, 280 nm and 230 nm were all elevated.

These results indicate that the absorbances at 420 nm, 280 nm and 230 nm could be largely lowered in the case of treating a saccharide solution of a solid concentration of 50% with active carbon, compared with the case of treating a saccharide solution of a solid concentration of 5% with active carbon and then concentrating to give a solid concentration of 50%.

TABLE 3
Absorbance of active carbon-treated saccharide solution
having solid concentration of 50% (1 cm cell)

| | 420 nm | | 280 nm | | 230 nm | |
|---|---|---|---|---|---|---|
| | | Removal | | Removal | | Removal |
| | Absor- | ratio | Absor- | ratio | Absor- | ratio |
| | bance | (%) | bance | (%)⁾ | bance | (%) |
| Before active | 0.142 | — | 6.13 | — | 12.15 | — |
| carbon treatment | | | | | | |
| After adding and | 0.05 | 64.8 | 1.7 | 72.3 | 4.99 | 58.9 |
| treating with 1% | | | | | | |
| active carbon | | | | | | |
| After adding and | 0.027 | 81.0 | 1.03 | 83.2 | 3.42 | 71.9 |
| treating with 2% | | | | | | |
| active carbon | | | | | | |
| After adding and | 0.007 | 95.1 | 0.58 | 90.5 | 2.28 | 81.2 |
| treating with 4% | | | | | | |
| active carbon | | | | | | |

Absorbance of active carbon-treated saccharide solution
having solid concentration of 5% (1 cm cell)

| | 420 nm | | 280 nm | | 230 nm | |
|---|---|---|---|---|---|---|
| | | Removal | | Removal | | Removal |
| | Absor- | ratio | Absor- | ratio | Absor- | ratio |
| | bance | (%) | bance | (%) | bance | (%)⁾ |
| Before active | 0.012 | — | 0.613 | — | 1.215 | — |
| carbon treatment | | | | | | |
| After adding and | 0.007 | 41.7 | 0.262 | 57.3 | 0.652 | 46.3 |
| treating with 0.1% | | | | | | |
| active carbon | | | | | | |
| After adding and | 0.004 | 66.7 | 0.182 | 70.3 | 0.516 | 57.5 |
| treating with 0.2% | | | | | | |
| active carbon | | | | | | |
| After adding and | 0.001 | 91.7 | 0.104 | 83.0 | 0.373 | 69.3 |
| treating with 0.4% | | | | | | |
| active carbon | | | | | | |
| After adding and | 0.007 | 41.7 | 0.397 | 35.2 | 0.778 | 36.0 |
| treating with 0.1% | | | | | | |
| active carbon, | | | | | | |
| heating at 100° C. | | | | | | |
| for 30 min | | | | | | |
| (Concentrated | 0.07 | — | 3.97 | — | 7.78 | — |
| 10-fold) | | | | | | |
| After adding and | 0.005 | 58.3 | 0.318 | 48.1 | 0.638 | 47.5 |
| treating with 0.2% | | | | | | |
| active carbon, | | | | | | |
| heating at 100° C. | | | | | | |
| for 30 min | | | | | | |
| (Concentrated | 0.05 | — | 3.18 | — | 6.38 | — |
| 10-fold) | | | | | | |
| After adding and | 0.004 | 66.7 | 0.239 | 61.0 | 0.515 | 57.6 |
| treating with 0.4% | | | | | | |
| active carbon, | | | | | | |
| heating at 100° C. | | | | | | |
| for 30 min | | | | | | |
| (Concentrated | 0.04 | — | 2.39 | — | 5.15 | — |
| 10-fold) | | | | | | |

### EXAMPLE 4

### Coloration Upon Heating Becoming Stronger with Increase in UV Absorption

Sample 1 was prepared by diluting the xylooligosaccharide syrup obtained in EXAMPLE 1 with purified water to give a saccharide concentration of 2%. The absorbances of sample 1 at 280 nm, 230 nm and 420 nm were respectively 0.043, 0.142 and 0.000. Sample 2 was prepared by heating sample 1 at 121° C. for 3 hours. The absorbances of sample 2 at 280 nm, 230 nm and 420 nm were respectively 7.72, 2.67 and 0.006, i.e., being almost colorless.

Sample 2 was further heated at 121° C. for 3 hours and the absorbances were measured. As a result, the absorbances thereof at 280 nm, 230 nm and 420 nm were respectively 15.62, 4.63 and 0.021, i.e., showing coloration in light brown.

It is understood that in the case where the absorbances at 280 nm and 230 nm exceeded 7.7 and 2.6, visible coloration was caused by the subsequent heating treatment at 121° C. for 3 hours.

TABLE 4

| | 420 nm | 280 nm | 230 nm |
|---|---|---|---|
| Before heating | 0.000 | 0.043 | 0.142 |
| Heated at 121° C. for 3 hours | 0.006 | 7.72 | 2.67 |
| Heated at 121° C. for 6 hours | 0.021 | 15.62 | 4.63 |
| Heated at 121° C. for 9 hours | 0.038 | 20.56 | 58.8 |

### EXAMPLE 5

### Production of Drink

A drink was produced by adding 4 g of the xylooligosaccharide syrup obtained in EXAMPLE 1 and 5 g of citric acid to 200 ml of water. The obtained drink had a refreshing sweetness.

### REFERENTIAL EXAMPLE 1

### Coloration Becoming Weaker with Increase in Polymerization Degree of Xylooligosaccharide

Xylose, xylobiose and xylotriose, each having a purity of 95% or higher, were dissolved in purified water to give a concentration of 2% by weight and then heated at 100° C. for 2 hours. The absorbances at 280 nm of these xylose, xylobiose and xylotriose solutions were respectively 0.257, 0.200 and 0.065, while the absorbances at 230 nm of these xylose, xylobiose and xylotriose solutions were respectively 0.791, 0.510 and 0.321. That is to say, the UV absorption showed less increase with an increase in the degree of polymerization. After heating at 121° C. for 6 hours, the absorbances of these solutions at 420 nm were measured to examine the coloration. As a result, the absorbances of the xylose, xylobiose and xylotriose solutions were respectively 0.031, 0.023 and 0.012. Namely, the coloration showed less increase with an increase in the degree of polymerization.

These results indicate that xylooligosaccharides containing xylobiose as the main component undergo stronger coloration due to heating than xylooligosaccharides containing as the main components xylooligosaccharides with polymerization degrees higher than that of xylobiose.

TABLE 5
Absorbance after heating of 2% by
weight solution in purified water

| | 420 nm | 280 nm | 230 nm |
|---|---|---|---|
| | (121° C., 6 hours) | (100° C., 2 hours) | (100° C., 2 hours) |
| Xylose | 0.031 | 0.257 | 0.791 |
| Xylobiose | 0.023 | 0.200 | 0.510 |
| Xylotriose | 0.012 | 0.065 | 0.321 |

1. A method of producing a high-purity xylooligosaccharide showing reduced UV absorption and/or reduced coloration which comprises alkali-treating or pressure- and heat-treating a plant material selected from the group consisting of corn cob, cotton seed hull, bagasse and rice straw, further enzymatically treating the same, concentrating the crude saccharide solution thus obtained followed by desalting and/or active carbon-treating, and, if necessary, drying the obtained saccharide solution to give a powder.

2. A method of producing a high-purity xylooligosaccharide showing reduced UV absorption and/or reduced coloration which comprises alkali-treating or pressure- and heat-treating a plant material selected from the group consisting of corn cob, cotton seed hull, bagasse and rice straw, further enzymatically treating the same, subjecting the thus obtained crude saccharide solution to desalting and/or active carbon-treating, then concentrating the crude saccharide solution, further conducting desalting and/or active carbon-treating, and, if necessary, drying the obtained saccharide solution to give a powder.

3. A method of producing a high-purity xylooligosaccharide showing reduced UV absorption and/or reduced coloration which comprises alkali-treating or pressure- and heat-treating a plant material selected from the group consisting of corn cob, cotton seed hull, bagasse and rice straw, further enzymatically treating the same, reacting the crude saccharide solution containing the residues thus obtained with lime and carbon dioxide gas to form an insoluble lime salt, removing the insoluble matters by filtration, concentrating the crude saccharide solution thus obtained followed by desalting and/or active carbon-treating, and, if necessary, drying the obtained saccharide solution to give a powder.

4. A method of producing a high-purity xylooligosaccharide containing reduced UV-absorbing substances and/or reduced coloration which comprises alkali-treating or pressure- and heat-treating a plant material selected from the group consisting of corn cob, cotton seed hull, bagasse and rice straw, further enzymatically treating the same, reacting the crude saccharide solution containing the residues thus obtained with lime and carbon dioxide gas to form an insoluble lime salt, removing the insoluble matters by filtration, subjecting the crude saccharide solution thus obtained to desalting and/or active carbon-treating followed by concentration, further conducting desalting and/or active carbon-treating, and if necessary, drying the obtained saccharide solution to give a powder.

5. A production method as claimed in claim 1 wherein the starting material is corn cob.

6. A production method as claimed in claim 1 whereby a xylooligosaccharide having a saccharide composition with a monosaccharide ratio of 30% or less is produced.

7. A production method as claimed in claim 6 whereby a xylooligosaccharide solution or a xylooligosaccharide powder showing, at a saccharide concentration of 37.5%, an absorbance at 280 nm in a 1 cm cell of 2 or less and/or an absorbance at 420 nm in a 5 cm cell of 0.2 or less is presented.

8. A production method as claimed in claim 1 whereby a xylooligosaccharide having a saccharide composition with a monosaccharide ratio of 5% or less is produced.

9. A production method as claimed in claim 8 whereby a xylooligosaccharide solution or a xylooligosaccharide powder showing, at a saccharide concentration of 20%, an absorbance at 280 nm in a 1 cm cell of 1 or less and/or an absorbance at 420 nm in a 5 cm cell of 0.1 or less is presented.

10. A production method as claimed in claim 1 whereby a xylooligosaccharide comprising xylobiose as the main component is produced.

11. A xylooligosaccharide solution or a xylooligosaccharide powder produced by a production method as claimed in claim 1 which has a saccharide composition with a monosaccharide ratio of 30% or less and shows, in the case of measuring at a saccharide concentration of 37.5%, an absorbance at 280 nm in a 1 cm cell of 2 or less and/or an absorbance at 420 nm in a 5 cm cell of 0.2 or less.

12. A xylooligosaccharide solution or a xylooligosaccharide powder produced by a production method as claimed in claim 1 which has a saccharide composition with a monosaccharide ratio of 5% or less and shows, in the case of measuring at a saccharide concentration of 20%, an absorbance at 280 nm in a 1 cm cell of 1 or less and/or an absorbance at 420 nm in a 5 cm cell of 0.1 or less.

13. A processed food, a drink, a health food, a supplement, a food for specified health uses, a cosmetic, a pet food or a drug containing a xylooligosaccharide produced by a method as claimed in claim 1.
